(19) United States
(12) Patent Application Publication
(10) Pub. No.: US 2026/0337546 A1
(43) Pub. Date: Oct. 29, 2026

(54) METHODS FOR IMPROVED CELL HARVESTING EFFICIENCY IN FIXED BED BIOREACTORS

(71) Applicant: CORNING INCORPORATED, CORNING, NY (US)

(72) Inventors: Li Cao, Hollis, NH (US); Vasiliy Nikolaevich Goral, Painted Post, NY (US); Yulong Hong, Boyds, MD (US)

(21) Appl. No.: 19/487,354

(22) PCT Filed: May 17, 2024
(86) PCT No.: PCT/US2024/029820

§ 371 Date: Nov. 24, 2025

Related U.S. Application Data
(60) Provisional application No. 63/470,173, filed on May 31, 2023.

(51) Int. Cl. C12N 1/02

(52) U.S. Cl. CPC C12N 1/02

(57) ABSTRACT

A method for harvesting cells from a bioreactor is provided where the bioreactor includes cells for harvesting, at least one inlet and/or outlet, and a culture chamber in which the cells are adhered to a substrate. The method includes providing a wash solution having a cell dissociation reagent (CDR) to the culture chamber; and after providing the wash solution, removing and capturing detached cells from the culture chamber via the at least one inlet and/ or outlet. The cell dissociation reagent includes an endonuclease, particularly a deoxyribonuclease, such as deoxyribonuclease I (DNase I). The wash solution may further include a divalent metal ion, such as Mg⁺², Ca⁺² or Mn⁺².

### CROSS-REFERENCE TO RELATED APPLICATIONS

This application claims the benefit of priority under 35 U.S.C. § 119 of U.S.

Provisional Application Ser. No. 63/470,173 filed on May 31, 2023, the content of which is relied upon and incorporated herein by reference in its entirety.

### FIELD OF THE DISCLOSURE

The present disclosure generally relates to the cell culturing and bioprocess field and, in particular, to a fixed-bed bioreactors and methods of using the same for culturing cells and harvesting cells from the bioreactor.

### BACKGROUND

In the bioprocessing industry, large scale cultivation of cells is performed for purposes of the production of hormones, enzymes, antibodies, vaccines and cell therapies.

Cell and gene therapies (CGT) are innovative technologies in modern medicine to treat diseases caused by genetic disorders. A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells. With more than 2,600 CGT clinical trials underway (see The Alliance for Regenerative Medicine: Regenerative Medicine in 2021: A Year of Firsts and Records, https://alliancerm.org/sector-report/h1-2021-report/), there is an unmet need for a robust manufacturing platform to meet the rapidly increasing clinical demands.

Methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing.

Another example of a high-density culture system for anchorage dependent cells is a packed bed bioreactor system. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, media flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of packed bed systems disclosed in a prior art is the inability to efficiently harvest intact viable cells at the end of culture process. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability. Contributing to the inefficiencies in harvesting are the often complex flow designs in packed bed bioreactors, where complicated flow paths make it difficult to efficiently removes cells from within the reactor without manually disassembling and harvesting the cells. The channeling and cell-trapping effects described above also decrease the potential for efficient cell harvesting.

In other current solutions available on the market, cells cannot be easily harvested from perfusion bioreactors using standard enzymatic methods. Instead, virus recovery via cell lysis with detergent is used, which adds time and complexity to down-stream purification of the virus.

Corning® has developed a novel fixed bed reactor (FBR) platform, the Corning® Ascent® FBR system, which combines the benefits of adherent bioproduction platforms with the scale and automation of suspension manufacturing systems. The systems are designed for use in CGT workflows such as, among others, adeno-associated virus (AAV), lentivirus (LV), mesenchymal stem cells (MSCs), and induced pluripotent stem cells (iPSCs) in addition to viral vaccine and other biologic production applications.

One of the key advantages of the Ascent® system is that viable cells can be aseptically harvested from the fixed-bed substrate. This ability to harvest viable cells enables the Ascent@ FBR system to be used as a seed train for manufacturing scale and production platform for cell therapy and regenerative medicine purposes. However, compared to cells cultured in a two-dimensional environment, cell harvesting from a fixed-bed bioreactor is challenging. Although the Corning® Ascent® system has demonstrated the capability of recovering viable cells from fixed bed reactor substrates, the harvest efficiency in fixed bed reactors systems generally can be improved, including in terms of consistency and especially for high density cell cultures. Lower cell harvest efficiency will result in the higher cost for both manufacturing and patients.

Thus, there remains a need for systems and method that enable consistent and efficient harvesting of adherent cells in fixed bed bioreactors.

### SUMMARY

According to embodiments of this disclosure, a method for harvesting cells from a bioreactor is provided. The method comprises providing the bioreactor having cells for harvesting. The bioreactor has at least one inlet and/or outlet, and a culture chamber in which the cells are adhered to a substrate. The method further includes providing a wash solution comprising a cell dissociation reagent (CDR) in the culture chamber. After providing the wash solution, the method further includes removing and capturing detached cells from the culture chamber via the at least one inlet and/or outlet. The cell dissociation reagent can include an endonuclease. The endonuclease can be a deoxyribonuclease, such as deoxyribonuclease I (DNase I). The wash solution can further include a divalent metal ion, such as Mg⁺², Ca⁺² or Mn⁺².

The method can further include providing a pre-harvest solution in the culture chamber; after providing the pre-harvest solution, providing a harvest solution in the culture chamber for a predetermined incubation time; following the predetermined incubation time, flushing the harvest solution from the culture chamber; and following the flushing of the harvest solution, providing a post-harvest solution in the culture chamber. At least one of the pre-harvest solution, the harvest solution, and the post-harvest solution can include the wash solution. The detached cells are harvested during the flushing of the harvest solution. The method can further include removing the post-harvest solution from the culture chamber and capturing residual cells removed by the post-harvest solution.

According to additional embodiments of this disclosure, a method for harvesting cells from a bioreactor is provided. The method includes providing the bioreactor comprising cells for harvesting, the bioreactor comprising an inlet, an outlet, and a culture chamber in which the cells are adhered to a substrate; providing a pre-harvest solution in the culture chamber; providing a harvest solution in the culture chamber for a predetermined incubation time; following the predetermined incubation time, flushing the harvest solution from the culture chamber; and following the flushing of the harvest solution, providing a post-harvest solution in the culture chamber. The detached cells are harvested during the flushing of the harvest solution.

The providing of the pre-harvest solution into the culture chamber can include removing cell culture media from the culture chamber. As an aspect of embodiments, the pre-harvest solution comprises a wash solution. The wash solution includes phosphate buffered-saline (PBS). The harvest solution can include a cell dissociation reagent (CDR). The cell dissociation reagent comprises at least one of a calcium chelator and a proteolytic enzyme. In aspects of embodiments, the flushing of the harvest solution from the culture chamber comprises using pressurized gas flowed into the culture chamber to displace the harvest solution. The flushing can also include removing cells that have been detached from the substrate. The method can include capturing the cells that have been detached and flushed from culture chamber in a harvest collection container. Providing the post-harvest solution in the culture chamber includes flowing the post-harvest solution the culture chamber. The method can further include capturing residual cells removed from the culture chamber by the post-harvest solution. The post-harvest solution can include PBS. At least one of the pre-harvest solution, the harvest solution, and the post-harvest solution comprises an endonuclease, such as deoxyribonuclease. The deoxyribonuclease can include deoxyribonuclease I (DNase I). The at least one of the pre-harvest solution, the harvest solution, and the post-harvest solution that comprises the endonuclease can further include a divalent metal ion, such as Mg⁺², Ca⁺² or Mn⁺².

Aspects of embodiments include mechanically agitating the culture chamber during harvesting. The mechanical agitation of the culture chamber occurs during at least one of the pre-harvest solution, the harvest solution, and the post-harvest solution being in the culture chamber. The mechanical agitation comprises at least one of stirring contents in the culture chamber, shaking or vibrating contents of the culture chamber, pulsing of fluid through the culture chamber, and altering a flow direction of fluid through the culture chamber.

Additional aspects of the present disclosure will be set forth, in part, in the detailed description, figures and any claims which follow, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

A more complete understanding of the present disclosure may be had by reference to the following detailed description when taken in conjunction with the accompanying drawings wherein:

FIG. 1A is a photograph showing an accumulation of biological material stretching between layers of a fixed bed substrate according to embodiments of the present disclosure;

FIG. 1B is a photograph showing a crystal-violet stained woven mesh substrate containing a DNA biofilm across the mesh and blocking the pores of the mesh, according to embodiments of the present disclosure;

FIG. 2A is a schematic illustrating a step of applying a pre-harvest solution to a fixed bed bioreactor, according to embodiments of the present disclosure;

FIG. 2B is a schematic illustrating a step of applying a harvest solution to a fixed bed bioreactor, according to embodiments of the present disclosure;

FIG. 2C is a schematic illustrating a step of applying a post-harvest solution to a fixed bed bioreactor, according to embodiments of the present disclosure;

FIG. 3 is a graph showing cell harvest yields when using no DNase and when using DNase in a post-harvest wash solution, according to embodiments of the present disclosure;

FIG. 4 is a graph showing cell harvest yields when using no DNase and when using a variety of DNase products in a post-harvest wash solution, according to embodiments of the present disclosure;

FIG. 5A is a flow chart showing a method of harvesting cells, according to embodiments of the present disclosure; and

FIG. 5B is a flow chart showing a method of harvesting cells, according to embodiments of the present disclosure.

### DETAILED DESCRIPTION

Various embodiments of the present disclosure will be discussed with reference to the figures, which illustrate various aspects of packed-bed bioreactor systems and related methods of using the bioreactor systems according to non-limiting embodiments of the present disclosure. The following description is intended to provide an enabling description of the bioreactor systems and the various aspects of the bioreactor systems and methods will be specifically discussed in detail throughout the disclosure with reference to the non-limiting embodiments, these embodiments are interchangeable with one another within the context of the disclosure.

According to embodiments of this disclosure, systems and methods for improved cell harvesting efficiencies when harvesting adherent cells from fixed bed bioreactors are provided. To harvest adherent cells, the cells growing on the surface of the substrate can be harvested using a cell dissociation reagent (CDR), according to aspects of embodiments.

CDR is used in adherent cell culture to detach cells from culture substrate. A component of a CDR is an enzyme with proteolytic activity. The enzyme digests the surface adhesion proteins and causes the separation of cells from the substrate. Trypsin is a commonly used CDR in cell passaging; however, excessive trypsinization can lead to irreversible cell damage. There are several trypsin alternative products developed for gentler detachment. Accutase® (by Innovative Cell Technologies, Inc.) is a natural enzyme mixture with proteolytic and collagenolytic enzyme activity. It is non-mammalian, non-bacterial reagent. TrypLE® (by Life Technologies Corporation) is a recombinant bacterial-derived animal origin-free product, and cGMP-compliant version is also available in the market. Both Accutase® and TrypLE® are very gentle to harvested cells.

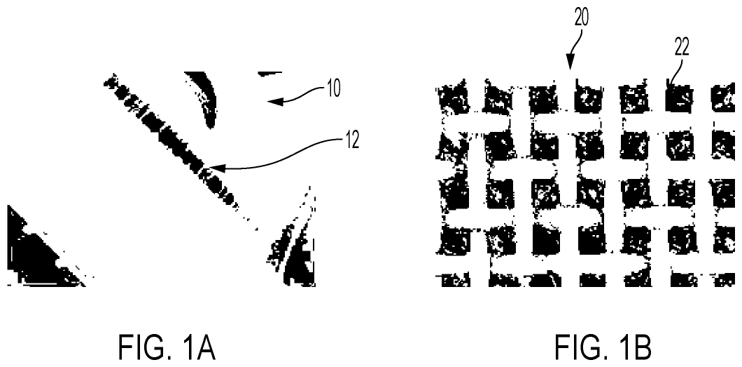

However, as discussed above, cell harvesting from a fixed-bed bioreactor is more challenging compared to a two-dimensional culture environment. Although bioreactors capable of recovering viable cells from FBR substrates have been demonstrated, the harvest efficiency could be more consistent, especially for high density cell culture. Experimental results have showed that cell harvest efficiencies for some fixed bed reactors (FBR) is not consistent using current harvest processes, with efficiencies in the range of 50-90%. It is believed that a possible root cause of low harvest efficiency may be due to the accumulation of DNA on the FBR substrate. The DNA can come from dead or low viability cells which could be introduced by inoculant or from cells damaged during the dynamic culture process (e.g., by shear force from fluid flow). This DNA circulating in the system can then attach to the substrate, as shown in FIG. 1A, with the most severe cases being on those substrates near the inlet of the FBR. This accumulation of DNA on FBR substrates not only can cause cells to form large aggregates, which may not pass through the substrate pores during the harvest process, but also can form biofilms that block pores in the substrate, as shown in FIG. 1B. The accumulation of aggregates and the blocking of openings in the substrate hampers cell and fluid flow uniformity, which can negatively impact not only havesting, but also the health and uniformity of the actual cell culture. As a result, the harvest efficiency was greatly compromised. This DNA problem is more severe in high density cell cultures.

As used herein, “harvest efficiency” refers to a percentage of cells in a cell culture run that can be harvested from the bioreactor by release and removal of the cells from the reactor in situ compared to the total number of cells present in the cell culture. The total number of cells present in the cell culture can be determined by adding the number of cells harvested with the number of cells detected in the bioreactor as determined by manually disassembling the bioreactor after the harvesting process is completed and crystal violet staining the substrate to determine the number of remaining cells on the substrate that were not successfully harvested.

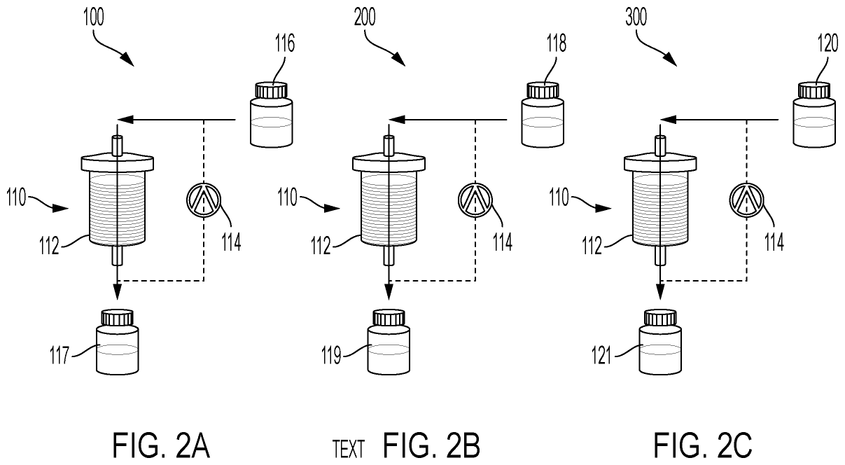

According to embodiments, a method of harvesting cells from a bioreactor is provided. As shown in FIG. 2A, a bioreactor 110 is provided that contains a cell culture substate 112 for culturing adherent cells. The bioreactor 110 includes an inlet and an outlet at opposite ends of the bioreactor 110 and substrate 112. Tubing connected to the inlet and outlet is used to perfuse cell culture media through the culture space within the bioreactor 110 that contains the substrate 112. In addition, one or more wash solutions (pre-harvest wash solution 116, harvest wash solution 118, and post-harvest wash solution 120) can be provided and connected to the tubing to supply wash solutions to the bioreactor 110. These wash solutions can be flowed through the bioreactor 110 and captured in a container 117, 119, 121 upon exiting the bioreactor 110, or can be perfused in a loop (e.g., in FIG. 2B during an incubation period using the harvest solution). One or more pumps 114 and valving may be used to control the flow path and rates. The process starts with a step 100 of flowing a pre-harvest solution 116, such as phosphate buffered saline (PBS) solution, through the bioreactor 110 to remove cell culture media. Following the pre-harvest solution wash, a cell harvest solution 118 is provided, such as Accutase® or TrypLE® (these are provided as examples only, and it is contemplated that any suitable CDR can be used). The cell harvest solution is pumped into the bioreactor 110 and incubated for a period to detach cells from the substrate 112. At the end of the predetermined incubation period, detached cells in bioreactor were harvested by flushing through an inlet or outlet of the bioreactor 110 and into a collection container 119 with the help of pressured air pumped into an opposite end of the bioreactor 110. Then, a post-harvest solution 120, such as PBS solution, is then flowed through the bioreactor 110 to recover residual detached cells.

As an aspect of embodiments, harvesting efficiency can be further improved by adding an endonuclease to one or more of the harvest solutions. For example, a deoxyribonuclease, such as deoxyribonuclease I (DNase I), can be added to any of the pre-harvest solution, the harvest solution, or the post-harvest solution. In one or more particular embodiments, the endonuclease is added to the post-harvest solution. DNase I is an endonuclease that hydrolyzes phosphodiester bonds and breaks or cleaves either double or single stranded DNA into oligonucleotides. DNase has been used in tissue digestion and single-cell suspension preparation. The purpose of addition of DNase to one of the harvest solutions in embodiments of this disclosure is to digest DNA accumulated on bioreactor substrate to improve harvest efficiency. As mentioned, in a particular embodiment, DNase I is added to the post-harvest solution; however, DNase I can be added to pre-harvest solution and/or cell harvest solution. Divalent metal ions, such as Mg⁺², Ca⁺² or Mn⁺², can also added to ensure full DNase I activity.

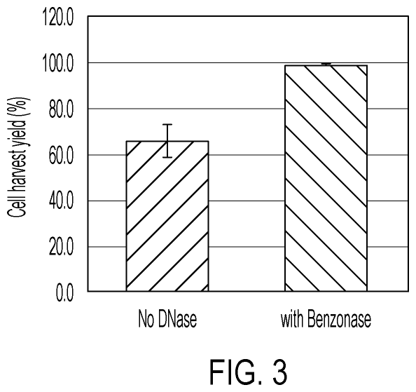

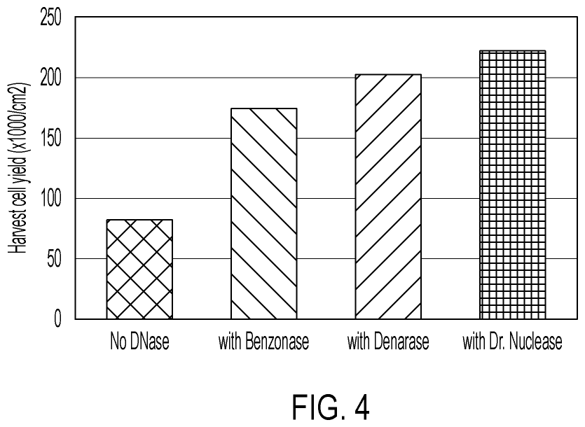

As shown in FIGS. 3 and 4, the use of DNase I in post-harvest wash solutions significantly improves cell harvest efficiencies when either Accutase® or TrypLE was used as a CDR. In FIG. 3, two experiments are compared where HEK293T cells were cultured in identical Corning® Ascent® FBR for three days each. The cells were then harvested without and with the addition of DNase to the post-harvest solution. The DNase product used is sold as Benzonase® (from MilliporeSigma). The experiment as repeated three times for each harvest condition, with the average cell harvest yield percentage shown by the columns in FIG. 3 and the spread as by range bars for each column. As shown in FIG. 4, DNase I from different vendors all gave similar, improved results. In each case, HEK293T cells were seeded at a density of 22,000 cells/cm² in Corning® Ascent® FBRs. After a three-day culture, cells were harvested with 1x TrypLE in the absence or presence of 25 U/mL of DNase I from three different venders in the post-harvest solution. The harvest cell yield is shown in x1000 cells/cm². Thus, according to embodiments of this disclosure, innovative cell harvesting processes are provided that can remove DNA accumulation on a bioreactor substrate, and therefore significantly improve cell harvest efficiency. DNase I can be added to the harvest process in different stages: pre-harvest wash solution, cell harvest solution, and/or post-harvest wash solution.

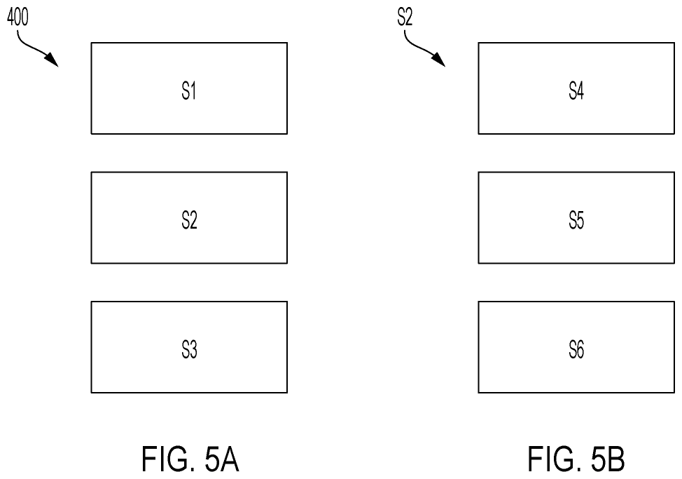

As shown in FIG. 5A, embodiments of this disclosure include a method 400 of harvest adherent cells from a fixed bed reactor that includes a step S1 of providing the bioreactor comprising cells for harvesting, a step S2 of providing a wash solution comprising a cell dissociation reagent in the culture chamber, and a step S3 of after providing the wash solution, removing and capturing detached cells from the culture chamber via the at least one inlet and/or outlet. The step S2 can include additional steps, such as the step S4 of providing a pre-harvest solution in the culture chamber; the step S5 of providing a harvest solution in the culture chamber for a predetermined incubation time; following the predetermined incubation time, flushing the harvest solution from the culture chamber; and the step S6 of, following the flushing of the harvest solution, providing a post-harvest solution in the culture chamber.

According to embodiments of this disclosure, a method for harvesting cells from a bioreactor can include providing the bioreactor comprising cells for harvesting. The bioreactor itself has at least one inlet and/or outlet, and a culture chamber in which the cells are adhered to a substrate. As an aspect of embodiments, the bioreactor has an inlet and an outlet on opposite sides of the bioreactor, with the substrate in between the inlet and outlet, so that fluid (e.g., cell culture media, nutrients, cells, and cell byproducts) can flow in a substantially linear direction from the inlet, through the substrate, and to the outlet. This simple linear flow path is believed to improve fluid flow uniformity, and thus cell culture uniformity and harvesting. However, embodiments are not intended to be limited to this arrangement and can include other flow designs.

The method also includes providing a wash solution that includes a cell dissociation reagent (CDR) in the culture chamber. The wash solution can be stored in a reservoir or container external to the bioreactor and supplied at a desired time to the culture chamber.

After providing the wash solution, detached cells are removed from the culture chamber via the at least one inlet and/or outlet and captured. The bioreactor system can include a harvest container fluidly connected to the at least one inlet and/or outlet for capturing the harvest cells. In an aspect to embodiments, the reactor has an inlet through which cell culture media is perfused in route to the substrate, and the cell culture media exits the bioreactor through the outlet. In further aspects of embodiments, the direction of flow through the bioreactor is reversed when supplying the wash solution. That is, the wash solution is stored in a container that is fluidly connected to the outlet of the bioreactor and is flowed into the culture chamber via the outlet and then into and/or through the substrate. The wash solution may also then flow out of the culture chamber through the inlet. Alternatively, the flow may be reversed again about some incubation time such that the wash solution flows out through the outlet.

According to further aspects of embodiments, the wash solution may be perfused in a recirculation loop through the cell culture chamber, such that the wash solution exits the bioreactor then is looped around to reenter the bioreactor, optionally after undergoing some type of reconditioning to maintain optimal properties of the wash solution. Alternatively, the wash solution can flow through the bioreactor only once, being captured in a container after existing the bioreactor.

According to additional aspects of embodiments, the cell dissociation reagent comprises a proteolytic enzyme, such as Accutase® or TrypLE®. The wash solution may further include an endonuclease, such as a deoxyribonuclease, for example. The deoxyribonuclease can include deoxyribonuclease I (DNase I). The wash solution can further include a divalent metal ion, such as Mg⁺², Ca⁺² or Mn⁺², for example.

In aspects of embodiments, the method can further include providing a pre-harvest solution in the culture chamber. The pre-harvest solution can include a phosphate buffered saline (PBS) solution. The pre-harvest solution is flowed into the bioreactor after the cell culture is completed to remove any cell culture media from the fixed bed substrate and to prepare the fixed bed for harvesting. The method can further include, after providing the pre-harvest solution, providing a harvest solution in the culture chamber for a predetermined incubation time. During this incubation time, a CDR in the harvest solution helps the cells detach from the substate so that they can be removed from the fixed bed. Following the predetermined incubation time, the harvest solution is flushed from the culture chamber. The flushing of the harvest solution acts to also flush the majority or entirety of the cells from the bioreactor. Following the flushing of the harvest solution, a post-harvest solution is provided in the culture chamber. The post-harvest solution can be used for an additional wash of the substrate and to harvest any additional cells that were not flushed with the harvest solution.

The method can further include removing the post-harvest solution from the culture chamber and capturing residual cells removed by the post-harvest solution. At least one of the pre-harvest solution, the harvest solution, and the post-harvest solution includes the wash solution mentioned above, according to embodiments.

According to embodiments of this disclosure, a method for harvesting cells from a bioreactor can include providing the bioreactor having cells for harvesting. The bioreactor includes an inlet, an outlet, and a culture chamber in which the cells are adhered to a substrate. The method also includes providing a pre-harvest solution in the culture chamber; providing a harvest solution in the culture chamber for a predetermined incubation time; following the predetermined incubation time, flushing the harvest solution from the culture chamber; and following the flushing of the harvest solution, providing a post-harvest solution in the culture chamber. The detached cells are harvested during the flushing of the harvest solution and, optionally, during a flushing of the post-harvest solution.

In aspects of embodiments, the providing of the pre-harvest solution into the culture chamber removes cell culture media from the culture chamber. The pre-harvest solution comprises a wash solution, such as phosphate buffered-saline (PBS), for example. Other accepted cell washing solutions may also be used, as well. Following providing the pre-harvest solution, the pre-harvest solution may be drained from the bioreactor prior to filling the bioreactor with the harvest solution, or the harvest solution may be supplied concurrently with removal of the pre-harvest solution. The harvest solution includes a cell dissociation reagent (CDR). The cell dissociation reagent comprises at least one of a calcium chelator and a proteolytic enzyme. According to aspects of embodiments, the flushing of the harvest solution from the culture chamber can include using pressurized gas flowed into the culture chamber to displace the harvest solution and force it out through one of the inlet and the outlet of the bioreactor. The flushing of the harvest solution also includes removing cells that have been detached from the substrate. Cells that have been detached and flushed are then captured in a harvest collection container.

In aspects of embodiments, the providing of the post-harvest solution in the culture chamber includes flowing the post-harvest solution into the culture chamber. If any cells remain in the fixed bed following flushing of the harvest solution, these residual cells can be removed from the culture chamber by the post-harvest solution and captured then. The postharvest solution can comprise a wash solution, such as PBS, or another accepted cell wash solution. As an aspect of embodiments, at least one of the pre-harvest solution, the harvest solution, and the post-harvest solution includes an endonuclease. The endonuclease can include a deoxyribonuclease, such as deoxyribonuclease I (DNase I), for example. In a further aspect, the at least one of the pre-harvest solution, the harvest solution, and the postharvest solution that includes the endonuclease further includes a divalent metal ion, such as Mg⁺², Ca⁺² or Mn⁺², for example. The presence of the divalent metal ion can increase the effectiveness of the endonuclease.

In further aspects of embodiments, the method can include mechanically agitating the culture chamber. The mechanical agitating of the culture chamber can occur during at least one of the pre-harvest solution, the harvest solution, and the post-harvest solution being in the culture chamber. The mechanical agitation can include at least one of stirring the contents in the culture chamber, shaking or vibrating contents of the culture chamber, pulsing of fluid through the culture chamber, and altering a flow direction of fluid through the culture chamber.

Referring to FIGS. 2A-2C, the fixed bed bioreactor 110 enables not only the culturing of adherent cells and harvesting of cell byproducts (e.g., transfected viruses), but also harvesting of the cells themselves. The cell culture substrate can take a variety of forms, but is preferably a porous material provided in a monolithic structure (such as a foam substrate or single woven mesh sheet) or in a packed-bed configuration of a plurality of pieces of substrate material or layers. In one embodiment, for example, the plurality of pieces of substrate material are sheets or discs of a porous polymer made from one or more polymer fibers. For example, the porous polymer material can be a woven mesh substrate material provided as a stack of sheets or discs within the vessel.

According to an aspect of embodiments, the bioreactor systems disclosed herein can pressurize the bioreactor and generate a safe flow rate that will not damage the cells or harvested product. The action of the pressurization for harvesting can be automated and fully integrated into a bioreactor controller system.

Current commercial products, which tend to use non-uniform substrates, trap cells nonuniformly in the substrate during attachment. This leads to a nonuniform cell growth and subsequent inefficient transfection of the DNA plasmids into the cells. This negatively impacts the total virus produced by the cells. In addition, because many of the cells become trapped, they cannot efficiently recover the virus inside the cells for later use in gene therapy. Currently as a work-around, detergents are used to lyse cells and release virus in situ; however, the addition of detergent adds cost and complexity to downstream virus purification steps. The embodiments of this disclosure eliminate the need for cell lysis, since the pressure delivered in combination with the various wash solutions can facilitate cell release from the bioreactor substrate. As an aspect of embodiments of this disclosure, a suitable cell culture substrate will enhance the flow uniformity and release of cells for harvesting. Examples of such substrate materials are disclosed in U.S. Pat. No. 11,434,460, the content of which is incorporated herein by reference in its entirety. The cell culture substrate is porous to allow perfusion of cells, media, nutrients, and cell by-products through the substrate and to allow spent media with cell secreted material (e.g., recombinant protein, antibody, virus particles, DNA, RNA, sugars, lipids, biodiesel, inorganic particles, butanol, metabolic byproducts) to pass through the substrate and be harvested. Further details of the cell culture substrate according to embodiments are provided below.

Embodiments of the present disclosure include bioreactors and cell culture substrates used therein, including substrates that are cell growth matrices and/or packed-bed systems for anchorage dependent cells that enable easy and effective scale-up to any practical production scale for cells or cell derived products (e.g., proteins, antibodies, viral particles). In one embodiment, a substrate is provided with a structurally defined surface area for adherent cells to attach and proliferate that has good mechanical strength and forms a highly uniform multiplicity of interconnected fluidic networks when assembled in a packed bed or other bioreactor. In particular embodiments, mechanically stable, non-degradable woven meshes can be used to support adherent cell production. Uniform cell seeding of such a substrate is achievable, as well as efficient harvesting of cells or other products of the bioreactor. In addition, the embodiments of this disclosure support cell culturing to achieve confluent monolayer or multilayer of adherent cells on the disclosed substrate, and can avoid formation of 3D cellular aggregates with limited nutrient diffusion and increased metabolite concentrations. The structurally defined substrate of one or more embodiments enables complete cell recovery and consistent cell harvesting from the packed bed of bioreactor. In another embodiment of the present disclosure, a method of cell culturing is provided using bioreactors with the substrate for bioprocessing production of therapeutic proteins, antibodies, viral vaccines, or viral vectors.

In one or more embodiments, the cell culture substrate supports attachment and proliferation of anchorage dependent cells in a high volumetric density format. The substrate can be assembled and used in a bioreactor system, such as a perfused fixed bed bioreactor as described herein, and provide uniform cell distribution during the inoculation step, while preventing formation of large and/or uncontrollable cell aggregates inside the fixed bed.

Thus, the substrate eliminates diffusional limitations during operation of the bioreactor. In addition, the substrate enables easy and efficient cell harvest from the bioreactor.

The substrate can be formed with a substrate material that of a thin, sheet-like construction having first and second sides separated by a relatively small thickness. In other words, the thickness of the sheet-like substrate is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; or a plurality of filaments that are woven into a mesh layer. The physical structure of the substrate has a high surface-to-volume ratio for culturing anchorage dependent cells.

According to various embodiments, the substrate can be arranged or packed in a bioreactor in certain ways to obtain uniform cell seeding, uniform media perfusion, and efficient cell harvest.

The cell culture substrate can be a woven mesh layer made of a first plurality of fibers running in a first direction and a second plurality of fibers running in a second direction. The woven fibers of the substrate form a plurality of openings. The size and shape of the openings can vary based on the type of weave (e.g., number, shape and size of filaments; angle between intersecting filaments, etc.). An opening can be defined by a certain width or diameter. A woven mesh may be considered, on a macro-scale, a two-dimensional sheet or layer. However, a close inspection of a woven mesh reveals a three-dimensional structure due to the rising and falling of intersecting fibers of the mesh. Thus, a thickness of the woven mesh may be thicker than the thickness of a single fiber.

The woven mesh can be comprised of monofilament or multifilament polymer fibers. In one or more embodiments, a monofilament fiber may have a diameter in a range of about 10 μm to about 1000 μm. On a microscale level, due to the scale of the fiber compared to the cells (e.g., the fiber diameters being larger than the cells), the surface of monofilament fiber is presented as regular 2D surface for adherent cells to attach and proliferate. Such fibers are woven into a mesh that has a defined pattern and a certain amount of structural rigidity. Fibers can be woven into a mesh with openings ranging from about 25 μm×25 μm to about 1000 μm×1000 μm. These ranges of the filament diameters and opening diameters are examples of some embodiments, but are not intended to limit the possible feature sizes of the mesh according to all embodiments.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different structure patterns or weaves, including, for example knitted, warp-knitted, or woven (plain weave, twilled weave, dutch weave, five needle weave).

The surface chemistry of the mesh filaments may need to be modified to provide desired cell adhesion properties. Such modifications can be made through the chemical treatment of the polymer material of mesh or grafting cell adhesion molecules to the filament surface. Alternatively, meshes can be coated with thin layer of biocompatible hydrogels that demonstrate cell adherence properties, including, for example, collagen or Matrigel®.

Alternatively, surfaces of filament fibers of the mesh can be rendered with cell adhesive properties through the treatment processes with various types of plasmas, process gases, and/or chemicals known in the industry.

The woven mesh substrate may be provided in a number of discs with a center hole configured to surround the center column of the bioreactor described herein. A plurality of such discs can be stacked in the outer region of the bioreactor to form the packed bed.

According to some embodiments, the cell culture substrate is a dissolvable foam scaffold comprising an ionotropically crosslinked polygalacturonic acid compound selected from at least one of: pectic acid; partially esterified pectic acid, partially amidated pectic acid and salts thereof; and at least one first water-soluble polymer having surface activity.

The embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase®.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A method for harvesting cells from a bioreactor, the method comprising:
providing the bioreactor comprising cells for harvesting, the bioreactor comprising at least one inlet and/or outlet, and a culture chamber in which the cells are adhered to a substrate;
providing a wash solution comprising a cell dissociation reagent (CDR) in the culture chamber; and
after providing the wash solution, removing and capturing detached cells from the culture chamber via the at least one inlet and/or outlet.

2. The method of claim 1, wherein the cell dissociation reagent comprises an endonuclease.

3. (canceled)

4. (canceled)

5. The method of claim 2, wherein the wash solution further comprises a divalent metal ion.

6. The method of claim 5, wherein the divalent metal ion comprises Mg⁺², Ca⁺² or Mn⁺².

7. The method of claim 1, further comprising:
providing a pre-harvest solution in the culture chamber;
after providing the pre-harvest solution, providing a harvest solution in the culture chamber for a predetermined incubation time;
following the predetermined incubation time, flushing the harvest solution from the culture chamber; and
following the flushing of the harvest solution, providing a post-harvest solution in the culture chamber,
wherein at least one of the pre-harvest solution, the harvest solution, and the postharvest solution comprises the wash solution.

8. (canceled)

9. The method of claim 7, wherein detached cells are harvested during the flushing of the harvest solution.

10. The method of claim 7, further comprising removing the post-harvest solution from the culture chamber and capturing residual cells removed by the post-harvest solution.

11. A method for harvesting cells from a bioreactor, the method comprising:
providing the bioreactor comprising cells for harvesting, the bioreactor comprising an inlet, an outlet, and a culture chamber in which the cells are adhered to a substrate;
providing a pre-harvest solution in the culture chamber, providing a harvest solution in the culture chamber for a predetermined incubation time;
following the predetermined incubation time, flushing the harvest solution from the culture chamber; and
following the flushing of the harvest solution, providing a post-harvest solution in the culture chamber,
wherein detached cells are harvested during the flushing of the harvest solution.

12. The method of claim 11, wherein the providing of the pre-harvest solution into the culture chamber comprises removing cell culture media from the culture chamber.

13. The method of claim 11, wherein the pre-harvest solution comprises a wash solution.

14. The method of claim 13, wherein the wash solution comprises phosphate buffered-saline (PBS).

15. The method of claim 11, wherein the harvest solution comprises a cell dissociation reagent (CDR).

16. (canceled)

17. The method of claim 11, wherein the flushing of the harvest solution from the culture chamber comprises using pressurized gas flowed into the culture chamber to displace the harvest solution.

18. The method of claim 11, wherein the flushing comprises removing cells that have been detached from the substrate.

19. The method of claim 18, further comprising capturing the cells that have been detached and flushed from culture chamber in a harvest collection container.

20. The method of claim 11, wherein providing the post-harvest solution in the culture chamber comprises flowing the post-harvest solution into the culture chamber.

21. The method of claim 11, further comprising capturing residual cells removed from the culture chamber by the post-harvest solution.

22. The method of claim 11, wherein the postharvest solution comprises PBS.

23. The method of claim 11, wherein at least one of the pre-harvest solution, the harvest solution, and the post-harvest solution comprises an endonuclease.

24. (canceled)

25. (canceled)

26. The method of claim 23, wherein the at least one of the pre-harvest solution, the harvest solution, and the post-harvest solution that comprises the endonuclease further comprises a divalent metal ion.

27-30. (canceled)
